(19) United States
(12) Patent Application Publication
(10) Pub. No.: US 2015/0368180 A1
(43) Pub. Date: Dec. 24, 2015

(54) ACID CATALYZED OLIGOMERIZATION OF ALKYL ESTERS AND CARBOXYLIC ACIDS

(71) Applicant: Elevance Renewable Sciences, Inc., Woodridge, IL (US)

(72) Inventors: Georgeta Hategan, Plainfield, IL (US); Steven A. Cohen, Naperville, IL (US); Ryan Littich, Shorewood, IL (US); Stephen A. Di Biase, River Forest, IL (US); Bruce Firth, Buffalo Grove, IL (US); Robin Weitkamp, Batavia, IL (US)

(73) Assignee: ELEVANCE RENEWABLE SCIENCES, INC., Woodridge, IL (US)

(21) Appl. No.: 14/840,874

(22) Filed: Aug. 31, 2015

Related U.S. Application Data
(63) Continuation of application No. 14/219,728, filed on Mar. 19, 2014.
(60) Provisional application No. 61/803,742, filed on Mar. 20, 2013.

(51) Int. Cl. C07C 69/608; C07C 69/604; C07C 57/02; C07C 57/13; C07C 57/26; C07C 69/593; C07C 69/602

(52) U.S. Cl. CPC C07C 69/608; C07C 69/593; C07C 69/604; C07C 69/602; C07C 57/13; C07C 57/26; C07C 57/02

(57) ABSTRACT

The oligomerization of certain carboxylic acids and alkyl esters derived from natural oils is disclosed. This includes the oligomerization of C₁₀₋₁₇ unsaturated carboxylic acids such as 9-decenoic acid, where the oligomerization yields a mixture of mono-, di- and tri-carboxylic acids. This also includes the oligomerization of certain alkyl esters, including the oligomerization of C₁₀₋₁₇ unsaturated alkyl esters such as methyl 9-decenoate (9-DAME), where the oligomerization yields a mixture of mono-, di- and tri-carboxylic acid esters. Various end use applications for the oligomerized carboxylic acids and oligomerized alkyl esters are also disclosed.

### CROSS REFERENCE TO RELATED APPLICATIONS

A claim of priority for this application under 35 U.S.C. §119(e) is hereby made to U.S. Non-Provisional patent application Ser. No. 14/219,728, filed Mar. 19, 2014, and U.S. Provisional Patent Application No. 61/803,742, filed Mar. 20, 2013; and these applications are incorporated herein by reference in their entireties.

### GOVERNMENT RIGHTS

This invention was made with government support under grant no. DE-EE0002872/001, awarded by the United States Department of Energy. The United States government has certain rights in this invention.

### FIELD OF THE INVENTION

The invention generally relates to acid catalyzed oligomerization of alkyl esters and carboxylic acids.

### BACKGROUND OF THE INVENTION

It is known that unsaturated carboxylic acids (including unsaturated fatty acids) and the alkyl esters of these carboxylic acids can be oligomerized to manufacture longer chain length dimerized, trimerized, or higher order oligomerized carboxylic acids and esters. Such oligomerization techniques have generally encompassed thermal oligomerization, and more commonly, acid-catalyzed oligomerization.

Acid catalyzed oligomerization is a cationic polymerization reaction. Cationic polymerization is a type of chain growth polymerization in which a cationic initiator transfers charge to a monomer which becomes reactive. This reactive monomer reacts with additional monomer to form a polymer. The solid acid-catalyzed initiators (clays, zeolites, ion exchange resin, and the like) typically require high temperature and only low molecular weight polymers are formed with these catalysts. Clay-catalyzed dimerization was developed and commercialized in the early 1950's by Emery Industries for the reaction of C₁₈ fatty acids and esters.

We have found that for the oligomerization of certain carboxylic acids, including the oligomerization of C₁₀₋₁₇ unsaturated carboxylic acids such as 9-decenoic acid, the oligomerization yields a mixture of mono-, di- and tri-carboxylic acids. We have also found that for the oligomerization of certain alkyl esters, including the oligomerization of C₁₀₋₁₇ unsaturated alkyl esters such as methyl 9-decenoate (9-DAME), the oligomerization yields a mixture of mono-, di- and tri-carboxylic acid esters. The monomer fraction of the reaction mixture is a mixture of positionally and skeletally isomerized monomers. After the isomerized monomer is removed by distillation, the polyfunctional esters mixture consists of dimers and trimers. In some embodiments, the weight ratio of dimer to trimer ranges from 20:80 to 80:20, and preferably in an 80:20 ratio. The mixture can also be further purified into pure dimer and trimer, or hydrogenated to yield products with lighter color and greater oxidative stability.

### BRIEF DESCRIPTION OF THE DRAWINGS

FIG. 1 depicts the effect of catalyst loading on selectivity of dimer (GC area %) at 190° C.

FIG. 2 depicts the effect of temperature on selectivity of dimer (GC area %) at 8 hours.

FIG. 3 depicts the effect of catalyst loading on selectivity of dimer (GC area %) at 160° C.

FIG. 4 depicts the effect of catalyst loading on selectivity of dimer (GC area %) at 220° C.

FIG. 5 depicts the effect of temperature on selectivity of dimer (GC area %) at 220° C. over time.

### SUMMARY OF THE INVENTION

In one aspect, a composition comprising a crude mixture of oligomers of metathesized C₁₀-C₁₇ alkyl esters is disclosed. The crude mixture comprises from about 18% to about 81% monomers of metathesized C₁₀-C₁₇ alkyl esters, from about 14% to about 46% dimers of metathesized C₁₀-C₁₇ alkyl esters, and from about from about 0% to about 18% trimers and/or higher unit oligomers of metathesized C₁₀-C₁₇ alkyl esters.

In another aspect, a composition comprising a crude mixture of oligomers of metathesized C₁₀-C₁₇ carboxylic acids is disclosed. The crude mixture comprises from about 30% to about 60% monomers of metathesized C₁₀-C₁₇ carboxylic acids, from about 30% to about 45% dimers of metathesized C₁₀-C₁₇ carboxylic acids, and from about 10% to about 25% trimers and/or higher unit oligomers of metathesized C₁₀-C₁₇ carboxylic acids.

### DETAILED DESCRIPTION OF THE INVENTION

It is to be understood that unless specifically stated otherwise, references to “a,” “an,” and/or “the” may include one or more than one, and that reference to an item in the singular may also include the item in the plural.

The term “natural oil” refers to oils or fats derived from plants or animals. The term “natural oil” includes natural oil derivatives, unless otherwise indicated, and such natural oil derivatives may include one or more natural oil derived unsaturated carboxylic acids or derivatives thereof. The natural oils may include vegetable oils, algae oils, fungus oils, animal oils or fats, tall oils, derivatives of these oils, combinations of two or more of these oils, and the like. The natural oils may include, for example, canola oil, rapeseed oil, coconut oil, corn oil, cottonseed oil, olive oil, palm oil, peanut oil, safflower oil, sesame oil, soybean oil, sunflower seed oil, linseed oil, palm kernel oil, tung oil, jatropha oil, mustard oil, camellina oil, pennycress oil, castor oil, coriander oil, almond oil, wheat germ oil, bone oil, lard, algal oil, tallow, poultry fat, yellow grease, fish oil, mixtures of two or more thereof, and the like. The natural oil (e.g., soybean oil) may be refined, bleached and/or deodorized. The natural oil may comprise a refined, bleached and/or deodorized natural oil, for example, a refined, bleached, and/or deodorized soybean oil (i.e., RBD soybean oil). The natural oil may also comprise a tall oil or an algal oil.

Natural oils of the type described herein typically are composed of triglycerides of fatty acids. These fatty acids may be either saturated, monounsaturated or polyunsaturated and contain varying chain lengths ranging from C₆ to C₃₀. These fatty acids may also be mono, di-, tri-, or poly-carboxylic acids. In some embodiments, the fatty acids may include hydroxy-substituted variants, aliphatic, cyclic, alicyclic, aromatic, branched, aliphatic- and alicyclic-substituted aromatic, aromatic-substituted aliphatic and alicyclic groups, saturated and unsaturated variants, and heteroatom substituted variants thereof. Some common fatty acids include saturated fatty acids such as lauric acid (dodecanoic acid), myristic acid (tetradecanoic acid), palmitic acid (hexadecanoic acid), stearic acid (octadecanoic acid), arachidic acid (eicosanoic acid), and lignoceric acid (tetracosanoic acid); unsaturated fatty acids as decenoic acid, undecenoic acid, dodecenoic acid, palmitoleic (a C16 acid), and oleic acid (a C18 acid); polyunsaturated acids include such fatty acids as linoleic acid (a di-unsaturated C18 acid), linolenic acid (a tri-unsaturated C18 acid), and arachidonic acid (a tetra-unsubstituted C20 acid).

The natural oils are further comprised of esters of these fatty acids in random placement onto the three sites of the trifunctional glycerine molecule. Such esters may be mono- or di-esters or poly-esters of these acids thereof. Different natural oils will have different ratios of these fatty acids, and within a given natural oil there is a range of these acids as well depending on such factors as where a vegetable or crop is grown, maturity of the vegetable or crop, the weather during the growing season, etc. Thus, it is difficult to have a specific or unique structure for any given natural oil, but rather a structure is typically based on some statistical average. For example soybean oil contains a mixture of stearic acid, oleic acid, linoleic acid, and linolenic acid in the ratio of 15:24:50:11, and an average number of double bonds of 4.4-4.7 per triglyceride. One method of quantifying the number of double bonds is the iodine value (IV) which is defined as the number of grams of iodine that will react with 100 grams of vegetable oil. Therefore for soybean oil, the average iodine value range is from 120-140. Soybean oil may comprises about 95% by weight or greater (e.g., 99% weight or greater) triglycerides of fatty acids. Major fatty acids in the polyol esters of soybean oil include saturated fatty acids, as a non-limiting example, palmitic acid (hexadecanoic acid) and stearic acid (octadecanoic acid), and unsaturated carboxylic acids, as a non-limiting example, oleic acid (9-octadecenoic acid), linoleic acid (9,12-octadecadienoic acid), and linolenic acid (9,12,15-octadecatrienoic acid).

The term “natural oil derivatives” refers to derivatives thereof derived from natural oil. The methods used to form these natural oil derivatives may include one or more of addition, neutralization, overbasing, saponification, transesterification, esterification, amidification, hydrogenation, isomerization, oxidation, alkylation, acylation, sulfurization, sulfonation, rearrangement, reduction, fermentation, pyrolysis, hydrolysis, liquefaction, anaerobic digestion, hydrothermal processing, gasification or a combination of two or more thereof. Examples of natural derivatives thereof may include carboxylic acids, gums, phospholipids, soapstock, acidulated soapstock, distillate or distillate sludge, fatty acids, fatty acid esters, as well as hydroxy substituted variations thereof, including unsaturated polyol esters. In some embodiments, the natural oil derivative may comprise an unsaturated carboxylic acid having from about 5 to about 30 carbon atoms, having one or more carbon-carbon double bonds in the hydrocarbon (alkene) chain. The natural oil derivative may also comprise an unsaturated fatty acid alkyl (e.g., methyl) ester derived from a glyceride of natural oil. For example, the natural oil derivative may be a fatty acid methyl ester (“FAME”) derived from the glyceride of the natural oil. In some embodiments, a feedstock includes canola or soybean oil, as a non-limiting example, refined, bleached, and deodorized soybean oil (i.e., RBD soybean oil).

The term “low-molecular-weight olefin” may refer to any one or combination of unsaturated straight, branched, or cyclic hydrocarbons in the C₂ to C₁₄ range. Low-molecular-weight olefins include “alpha-olefins” or “terminal olefins,” wherein the unsaturated carbon-carbon bond is present at one end of the compound. Low-molecular-weight olefins may also include dienes or trienes. Examples of low-molecular-weight olefins in the C₂ to C₆ range include, but are not limited to: ethylene, propylene, 1-butene, 2-butene, isobutene, 1-pentene, 2-pentene, 3-pentene, 2-methyl-1-butene, 2-methyl-2-butene, 3-methyl-1-butene, cyclopentene, 1-hexene, 2-hexene, 3-hexene, 4-hexene, 2-methyl-1-pentene, 3-methyl-1-pentene, 4-methyl-1-pentene, 2-methyl-2-pentene, 3-methyl-2-pentene, 4-methyl-2-pentene, 2-methyl-3-pentene, and cyclohexene. Other possible low-molecular-weight olefins include styrene and vinyl cyclohexane. In certain embodiments, it is preferable to use a mixture of olefins, the mixture comprising linear and branched low-molecular-weight olefins in the C₄-C₁₀ range. In one embodiment, it may be preferable to use a mixture of linear and branched C₄ olefins (i.e., combinations of: 1-butene, 2-butene, and/or isobutene). In other embodiments, a higher range of C₁₁-C₁₄ may be used.

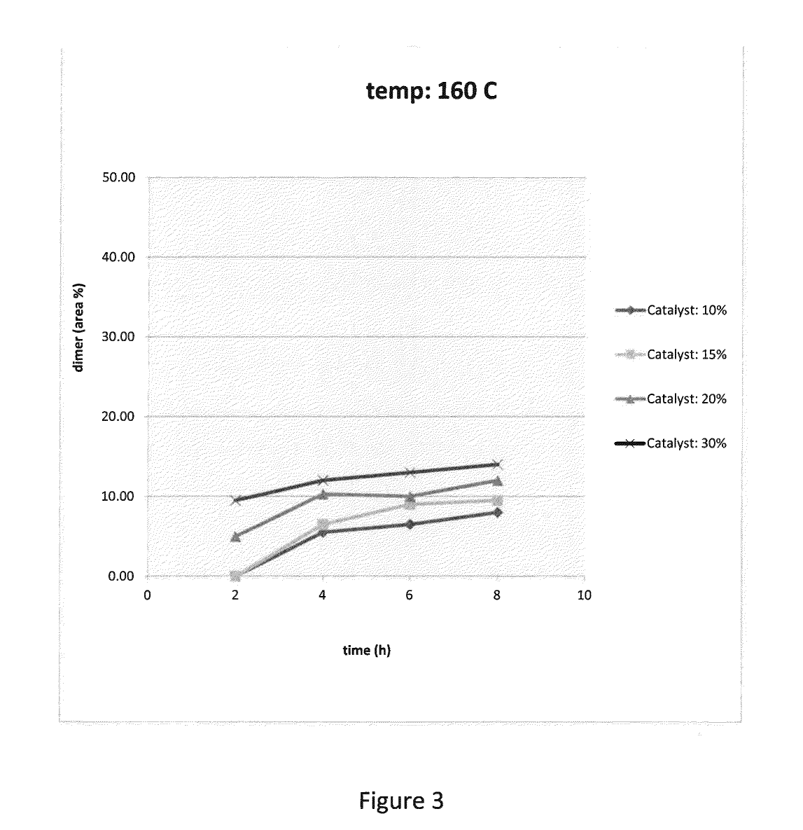

As used herein, the terms “metathesize” and “metathesizing” may refer to the reacting of a natural oil feedstock in the presence of a metathesis catalyst to form a metathesized natural oil product comprising a new olefinic compound and/or esters. Metathesizing may refer to cross-metathesis (a.k.a. co-metathesis), self-metathesis, ring-opening metathesis, ring-opening metathesis polymerizations (“ROMP”), ring-closing metathesis (“RCM”), and acyclic diene metathesis (“ADMET”). As a non-limiting example, metathesizing may refer to reacting two triglycerides present in a natural feedstock (self-metathesis) in the presence of a metathesis catalyst, wherein each triglyceride has an unsaturated carbon-carbon double bond, thereby forming an oligomer having a new mixture of olefins and esters that may comprise one or more of: metathesis monomers, metathesis dimers, metathesis trimers, metathesis tetramers, metathesis pentamers, and higher order metathesis oligomers (e.g., metathesis hexamers, metathesis, metathesis heptamers, metathesis octamers, metathesis nonamers, metathesis decamers, and higher than metathesis decamers and above). In some aspects, a metathesis dimer refers to a compound formed when two unsaturated polyol ester molecules are covalently bonded to one another by a self-metathesis reaction, and a metathesis trimer refers to a compound formed when three unsaturated polyol ester molecules are covalently bonded together by metathesis reactions. In some aspects, a metathesis trimer is formed by the cross-metathesis of a metathesis dimer with an unsaturated polyol ester. In some aspects, a metathesis tetramer refers to a compound formed when four unsaturated polyol ester molecules are covalently bonded together by metathesis reactions. In some aspects, a metathesis tetramer is formed by the cross-metathesis of a metathesis trimer with an unsaturated polyol ester. Metathesis tetramers also may be formed, for example, by the cross-metathesis of two metathesis dimers. Higher unit metathesis products also may be formed. For example, metathesis pentamers and metathesis hexamers also may be formed. In some embodiments, metathesis reactions are commonly accompanied by isomerization, which may or may not be desirable. See, for example, G. Djigoué and M. Meier, Appl. Catal. A 346 (2009) 158, especially FIG. 3. Thus, the skilled person might modify the reaction conditions to control the degree of isomerization or alter the proportion of cis- and trans-isomers generated. For instance, heating a metathesis product in the presence of an inactivated metathesis catalyst might allow the skilled person to induce double bond migration to give a lower proportion of product having trans-Δ⁹ geometry.

The term “metathesis catalyst” includes any catalyst or catalyst system that catalyzes a metathesis reaction. Any known metathesis catalyst may be used, alone or in combination with one or more additional catalysts. Suitable homogeneous metathesis catalysts include combinations of a transition metal halide or oxo-halide (e.g., WOCl₄ or WCl₆) with an alkylating cocatalyst (e.g., Me₄Sn), or alkylidene (or carbene) complexes of transition metals, particularly Ru, Mo, or W. These include first and second-generation Grubbs catalysts, Grubbs-Hoveyda catalysts, and the like. Suitable alkylidene catalysts have the general structure:

M[X¹X²L¹L²(L³)ₙ]=Cₘ═C(R¹)R²

where M is a Group 8 transition metal, L¹, L², and L³ are neutral electron donor ligands, n is 0 (such that L³ may not be present) or 1, m is 0, 1, or 2, X¹ and X² are anionic ligands, and R¹ and R² are independently selected from H, hydrocarbyl, substituted hydrocarbyl, heteroatom-containing hydrocarbyl, substituted heteroatom-containing hydrocarbyl, and functional groups. Any two or more of X¹, X², L¹, L², L³, R¹ and R² can form a cyclic group and any one of those groups can be attached to a support.

First-generation Grubbs catalysts fall into this category where m=n=0 and particular selections are made for n, X¹, X², L¹, L², L³, R¹ and R² as described in U.S. Pat. Appl. Publ. No. 2010/0145086, the teachings of which related to all metathesis catalysts are incorporated herein by reference.

Second-generation Grubbs catalysts also have the general formula described above, but L¹ is a carbene ligand where the carbene carbon is flanked by N, O, S, or P atoms, preferably by two N atoms. Usually, the carbene ligand is part of a cyclic group. Examples of suitable second-generation Grubbs catalysts also appear in the '086 publication.

In another class of suitable alkylidene catalysts, L¹ is a strongly coordinating neutral electron donor as in first- and second-generation Grubbs catalysts, and L² and L³ are weakly coordinating neutral electron donor ligands in the form of optionally substituted heterocyclic groups. Thus, L² and L³ are pyridine, pyrimidine, pyrrole, quinoline, thiophene, or the like.

In yet another class of suitable alkylidene catalysts, a pair of substituents is used to form a bi- or tridentate ligand, such as a biphosphine, dialkoxide, or alkyldiketonate. Grubbs-Hoveyda catalysts are a subset of this type of catalyst in which L² and R² are linked. Typically, a neutral oxygen or nitrogen coordinates to the metal while also being bonded to a carbon that is α-, β-, or γ—with respect to the carbene carbon to provide the bidentate ligand. Examples of suitable Grubbs-Hoveyda catalysts appear in the '086 publication.

The structures below provide just a few illustrations of suitable catalysts that may be used:

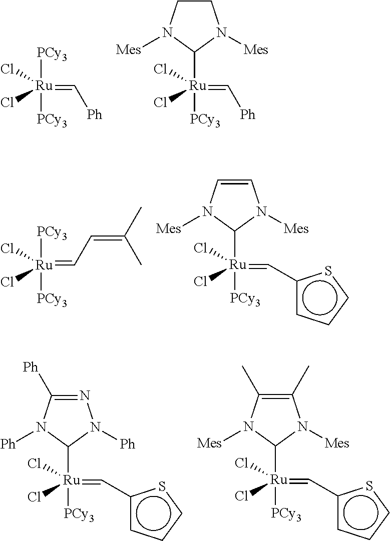

Heterogeneous catalysts suitable for use in the self- or cross-metathesis reaction include certain rhenium and molybdenum compounds as described, e.g., by J. C. Mol in Green Chem. 4 (2002) 5 at pp. 11-12. Particular examples are catalyst systems that include Re₂O₇ on alumina promoted by an alkylating cocatalyst such as a tetraalkyl tin lead, germanium, or silicon compound. Others include MoCl₃ or MoCl₅ on silica activated by tetraalkyltins.

For additional examples of suitable catalysts for self- or cross-metathesis, see U.S. Pat. Nos. 4,545,941, 5,312,940, 5,342,909, 5,710,298, 5,728,785, 5,728,917, 5,750,815, 5,831,108, 5,922,863, 6,306,988, 6,414,097, 6,696,597, 6,794,534, 7,102,047, 7,378,528, and U.S. Pat. Appl. Publ. No. 2009/0264672 A1, and PCT/US2008/009635, pp. 18-47, all of which are incorporated herein by reference. A number of metathesis catalysts that may be advantageously employed in metathesis reactions are manufactured and sold by Materia, Inc. (Pasadena, Calif.).

### Methods for Making Alkyl Esters and Carboxylic Acids.

The methods for generating alkyl esters and fatty acids are by transesterification or hydrolysis of triglycerides from a natural oil. Such alkyl esters and carboxylic acids, either individually or in combination, are subject to subsequent oligomerization as described later in this document. The self-metathesis of unsaturated alkyl esters can provide an equilibrium mixture of starting material, an internally unsaturated hydrocarbon, and an unsaturated diester. For instance, methyl oleate(methyl cis-9-octadecenoate) is partially converted to 9-octadecene and dimethyl 9-octadecenedioate, with both products consisting predominantly of the trans-isomer. Metathesis effectively isomerizes the cis-double bond of methyl oleate to give an equilibrium mixture of cis- and trans-isomers in both the “unconverted” starting material and the metathesis products, with the trans-isomers predominating. Cross-metathesis of unsaturated alkyl esters with low molecular olefins generates new olefins and new unsaturated alkyl esters that can have reduced chain length. For instance, cross-metathesis of methyl oleate and 3-hexene provides 3-dodecene and methyl 9-dodecenoate (see also U.S. Pat. No. 4,545,941). A variety of cross-metathesis reactions involving an α-olefin and an unsaturated alkyl ester (as the internal olefin source) are described. Thus, for example, reaction of soybean oil with propylene followed by hydrolysis gives, among other things, 1-decene, 2-undecenes, 9-decenoic acid, and 9-undecenoic acid.

In particular, the alkyl esters and carboxylic acids may be generated as follows. After an optional treatment of the natural oil feedstock (which may include thermal and/or chemical, and/or adsorbent methods to remove catalyst poisons, or a partial hydrogenation treatment to modify the natural oil feedstock's reactivity with the metathesis catalyst), the natural oil is reacted with itself, or combined with a low-molecular-weight olefin in a metathesis reactor in the presence of a metathesis catalyst. In certain embodiments, in the presence of a metathesis catalyst, the natural oil undergoes a self-metathesis reaction with itself. In other embodiments, in the presence of the metathesis catalyst, the natural oil undergoes a cross-metathesis reaction with the low-molecular-weight olefin. In certain embodiments, the natural oil undergoes both self- and cross-metathesis reactions in parallel metathesis reactors. Multiple, parallel, or sequential metathesis reactions (at least one or more times) may be conducted. The self-metathesis and/or cross-metathesis reaction form a metathesized natural oil product wherein the metathesized natural oil product comprises olefins and esters. In some embodiments, metathesized natural oil product is metathesized soybean oil (MSBO).

In another embodiment, the low-molecular-weight olefin comprises at least one branched low-molecular-weight olefin in the C₄ to C₁₀ range. Non-limiting examples of branched low-molecular-weight olefins include isobutene, 3-methyl-1-butene, 2-methyl-3-pentene, and 2,2-dimethyl-3-pentene. By using these branched low-molecular-weight olefins in the metathesis reaction, the metathesized natural oil product will include branched olefins, which can be subsequently hydrogenated to iso-paraffins. In certain embodiments, the branched low-molecular-weight olefins may help achieve the desired performance properties for a fuel composition, such as jet, kerosene, or diesel fuel.

As noted, it is possible to use a mixture of various linear or branched low-molecular-weight olefins in the reaction to achieve the desired metathesis product distribution. In one embodiment, a mixture of butenes (1-butene, 2-butenes, and, optionally, isobutene) may be employed as the low-molecular-weight olefin, offering a low cost, commercially available feedstock instead a purified source of one particular butene. Such low cost mixed butene feedstocks are typically diluted with n-butane and/or isobutane.

In certain embodiments, recycled streams from downstream separation units may be introduced to the metathesis reactor in addition to the natural oil and, in some embodiments, the low-molecular-weight olefin. For instance, in some embodiments, a C₂-C₆ recycle olefin stream or a C₃-C₄ bottoms stream from an overhead separation unit may be returned to the metathesis reactor. In one embodiment a light weight olefin stream from an olefin separation unit may be returned to the metathesis reactor. In another embodiment, the C₃-C₄ bottoms stream and the light weight olefin stream are combined together and returned to the metathesis reactor. In another embodiment, a C₁₅₊ bottoms stream from the olefin separation unit is returned to the metathesis reactor. In another embodiment, all of the aforementioned recycle streams are returned to the metathesis reactor.

The metathesis reaction in the metathesis reactor produces a metathesized natural oil product. In one embodiment, the metathesized natural oil product enters a flash vessel operated under temperature and pressure conditions which target C₂ or C₂-C₃ compounds to flash off and be removed overhead. The C₂ or C₂-C₃ light ends are comprised of a majority of hydrocarbon compounds having a carbon number of 2 or 3. In certain embodiments, the C₂ or C₂-C₃ light ends are then sent to an overhead separation unit, wherein the C₂ or C₂-C₃ compounds are further separated overhead from the heavier compounds that flashed off with the C₂-C₃ compounds. These heavier compounds are typically C₃-C₅ compounds carried overhead with the C₂ or C₂-C₃ compounds. After separation in the overhead separation unit, the overhead C₂ or C₂-C₃ stream may then be used as a fuel source. These hydrocarbons have their own value outside the scope of a fuel composition, and may be used or separated at this stage for other valued compositions and applications. In certain embodiments, the bottoms stream from the overhead separation unit containing mostly C₃-C₅ compounds is returned as a recycle stream to the metathesis reactor. In the flash vessel, the metathesized natural oil product that does not flash overhead is sent downstream for separation in a separation unit, such as a distillation column.

Prior to the separation unit, in certain embodiments, the metathesized natural oil product may be introduced to an adsorbent bed to facilitate the separation of the metathesized natural oil product from the metathesis catalyst. In one embodiment, the adsorbent is a clay bed. The clay bed will adsorb the metathesis catalyst, and after a filtration step, the metathesized natural oil product can be sent to the separation unit for further processing. Separation unit may comprise a distillation unit. In some embodiments, the distillation may be conducted, for example, by steam stripping the metathesized natural oil product. Distilling may be accomplished by sparging the mixture in a vessel, typically agitated, by contacting the mixture with a gaseous stream in a column that may contain typical distillation packing (e.g., random or structured), by vacuum distillation, or evaporating the lights in an evaporator such as a wiped film evaporator. Typically, steam stripping will be conducted at reduced pressure and at temperatures ranging from about 100° C. to 250° C. The temperature may depend, for example, on the level of vacuum used, with higher vacuum allowing for a lower temperature and allowing for a more efficient and complete separation of volatiles.

In another embodiment, the adsorbent is a water soluble phosphine reagent such as tris hydroxymethyl phosphine (THMP). Catalyst may be separated with a water soluble phosphine through known liquid-liquid extraction mechanisms by decanting the aqueous phase from the organic phase. In other embodiments, the metathesized natural oil product may be contacted with a reactant to deactivate or to extract the catalyst.

In the separation unit, in certain embodiments, the metathesized natural oil product is separated into at least two product streams. In one embodiment, the metathesized natural oil product is sent to the separation unit, or distillation column, to separate the olefins from the esters. In another embodiment, a byproduct stream comprising C₇'s and cyclohexadiene may be removed in a side-stream from the separation unit. In certain embodiments, the separated olefins may comprise hydrocarbons with carbon numbers up to 24. In certain embodiments, the esters may comprise metathesized glycerides. In other words, the lighter end olefins are preferably separated or distilled overhead for processing into olefin compositions, while the esters, comprised mostly of compounds having carboxylic acid/ester functionality, are drawn into a bottoms stream. Based on the quality of the separation, it is possible for some ester compounds to be carried into the overhead olefin stream, and it is also possible for some heavier olefin hydrocarbons to be carried into the ester stream.

In one embodiment, the olefins may be collected and sold for any number of known uses. In other embodiments, the olefins are further processed in an olefin separation unit and/or hydrogenation unit (where the olefinic bonds are saturated with hydrogen gas). In other embodiments, esters comprising heavier end glycerides and free fatty acids are separated or distilled as a bottoms product for further processing into various products. In certain embodiments, further processing may target the production of the following non-limiting examples: fatty acid methyl esters; biodiesel; 9DA (9-decenoic acid) esters, 9UDA (9-undecenoic acid) esters, 10UDA (10-undecenoic) esters and/or 9DDA (9-dodecenoic acid) esters; 9DA (9-decenoic acid), 9UDA (9-undecenoic acid), 10UDA (10-undecenoic acid) and/or 9DDA (9-dodecenoic acid); alkali metal salts and alkaline earth metal salts of 9DA, 9UDA, and/or 9DDA; diacids, and/or diesters of the transesterified products; and mixtures thereof. In certain embodiments, further processing may target the production of C₁₃-C₁₇ carboxylic acids and/or esters. In other embodiments, further processing may target the production of diacids and/or diesters. In yet other embodiments, further processing may target the production of compounds having molecular weights greater than the molecular weights of stearic acid and/or linolenic acid.

With regard to the esters from the distillation unit, in certain embodiments, the esters may be entirely withdrawn as an ester product stream and processed further or sold for its own value. Based upon the quality of separation between olefins and esters, the esters may comprise some heavier olefin components carried with the triglycerides. In other embodiments, the esters may be further processed in a biorefinery or another chemical or fuel processing unit known in the art, thereby producing various products such as biodiesel or specialty chemicals that have higher value than that of the triglycerides, for example. Alternatively, in certain embodiments, the esters may be partially withdrawn from the system and sold, with the remainder further processed in the biorefinery or another chemical or fuel processing unit known in the art.

In certain embodiments, the ester stream is sent to a transesterification unit. Within the transesterification unit, the esters are reacted with at least one alcohol in the presence of a transesterification catalyst. In certain embodiments, the alcohol comprises methanol and/or ethanol. In one embodiment, the transesterification reaction is conducted at approximately 60-70° C. and approximately 1 atm. In certain embodiments, the transesterification catalyst is a homogeneous sodium methoxide catalyst. Varying amounts of catalyst may be used in the reaction, and, in certain embodiments, the transesterification catalyst is present in the amount of approximately 0.5-1.0 weight % of the esters.

The transesterification reaction may produce transesterified products including saturated and/or unsaturated fatty acid methyl esters (“FAME”), glycerin, methanol, and/or free fatty acids. In certain embodiments, the transesterified products, or a fraction thereof, may comprise a source for biodiesel. In certain embodiments, the transesterified products comprise 9DA (9-decenoic acid) esters, 9UDA (9-undecenoic acid), 10UDA (10-undecenoic acid) esters, and/or 9DDA (9-dodecenoic acid) esters. Non-limiting examples of 9DA esters, 9UDA esters and 9DDA esters include methyl 9-decenoate (“9-DAME”), methyl 10-undecenoate (“10-UDAME”), and methyl 9-dodecenoate (“9-DDAME”), respectively. In some embodiments, the transesterified products may including C₁₃-C₁₇ unsaturated alkyl esters, including esters derived from 9-tridecenoic acid, 9-tetradecenoic acid, 9-pentadecenoic acid, 9-hexadecenoic acid, 9-heptadecenoic acid, and the like. As a non-limiting example, in a transesterification reaction, a 9DA moiety of a metathesized glyceride is removed from the glycerol backbone to form a 9DA ester.

In another embodiment, a glycerin alcohol may be used in the reaction with a glyceride stream. This reaction may produce monoglycerides and/or diglycerides. In certain embodiments, the transesterified products from the transesterification unit can be sent to a liquid-liquid separation unit, wherein the transesterified products (i.e., FAME, free fatty acids, and/or alcohols) are separated from glycerin. Additionally, in certain embodiments, the glycerin byproduct stream may be further processed in a secondary separation unit, wherein the glycerin is removed and any remaining alcohols are recycled back to the transesterification unit for further processing.

In one embodiment, the transesterified products are further processed in a water-washing unit. In another embodiment, the water-washing step is followed by a drying unit in which excess water is further removed from the desired mixture of esters (i.e., specialty chemicals). Such specialty chemicals include non-limiting examples such as 9DA (9-decenoic acid), 9UDA (9-undecenoic acid), 10UDA (10-undecenoic acid), and/or 9DDA (9-dodecenoic acid), alkali metal salts and alkaline earth metal salts of the preceding, individually or in combinations thereof.

In one embodiment, the specialty chemical (e.g., 9DA) may be further processed in an oligomerization reaction to form a lactone, which may serve as a precursor to a surfactant.

In certain embodiments, the transesterified products from the transesterification unit or specialty chemicals from the water-washing unit or drying unit are sent to an ester distillation column for further separation of various individual or groups of compounds. In one embodiment, under known operating conditions, the 9DA ester, 9UDA ester, 10UDA ester, 9DDA and/or C₁₃-C₁₇ unsaturated alkyl esters may then undergo a hydrolysis reaction with water yielding free fatty acids and glycerol as the product, where such free fatty acids are 9DA, 9UDA, 10UDA, 9DDA, C₁₃-C₁₇ unsaturated fatty acids, alkali metal salts and alkaline earth metal salts of the preceding, individually or in combinations thereof.

In certain embodiments, the fatty acid methyl esters (i.e. 9DA ester, 9UDA ester, 10UDA ester, 9DDA and/or C₁₃-C₁₇ unsaturated alkyl esters) from the transesterified products may be reacted with each other to form other specialty chemicals such as oligomerized esters, such as dimers, timer, tetramer, pentamer or higher esters. In some embodiments, 9DA, 9UDA, 10UDA, 9DDA and/or C₁₃-C₁₇ unsaturated fatty acids may be reacted with each other to form other specialty chemicals such as oligomerized acids, such as dimers, trimer, tetramer, pentamer or higher acids. In some embodiments, the fatty acid methyl esters and unsaturated fatty acids may be reacted with each other to produce oligomerized esters and/or acids. In some embodiments, C₁₈ unsaturated fatty acids such as oleic, linoleic and linolenic acids, often found in commercially available tall oils, may be reacted with the fatty acid methyl esters and/or unsaturated fatty acids. Generally, monounsaturated fatty acids (e.g., oleic acid) generally dimerize via electrophilic addition-elimination. Diunsaturated and triunsaturated fatty acids (e.g., linoleic, linolenic acid) dimerize by electrophilic addition-elimination, but also by [4+2] cycloaddition. The conditions under which dimerization/oligomerization is performed will give rise to a number of alkylation and olefin regioisomers as reaction products. Different points of carbon-carbon bond formation and unsaturation are expected.

In some embodiments, the unsaturated fatty acid may be a C18 diacid such as 9-octadecenedioic acid (9-ODDA), which can be generated by the metathesis of 9DA and/or 9DDA. In some embodiments, the unsaturated alkyl ester is a C18 diester such as dimethyl 9-octadecenedioate (9-ODDAME), which can be generated by the self metathesis of methyl oleate. The 9-ODDAME could be produced by: (i) cross-metathesis of 9-DAME with 9-DDAME to form cis/trans 9-ODDAME and 1-butene; (ii) cross-metathesis of 9-DAME with 9-UDAME to form cis/trans 9-ODDAME and 1-propene; (iii) self-metathesis of 9-DDAME to form cis/trans 9-ODDAME and 3-hexene; and (iv) self-metathesis of 9-UDAME to form cis/trans 9-ODDAME and 2-butene.

Some additional non-limiting examples of carboxylic acid dimers from these biorefinery monomers are shown in the structures below. The corresponding esters of these acids are also inferred, though not shown.

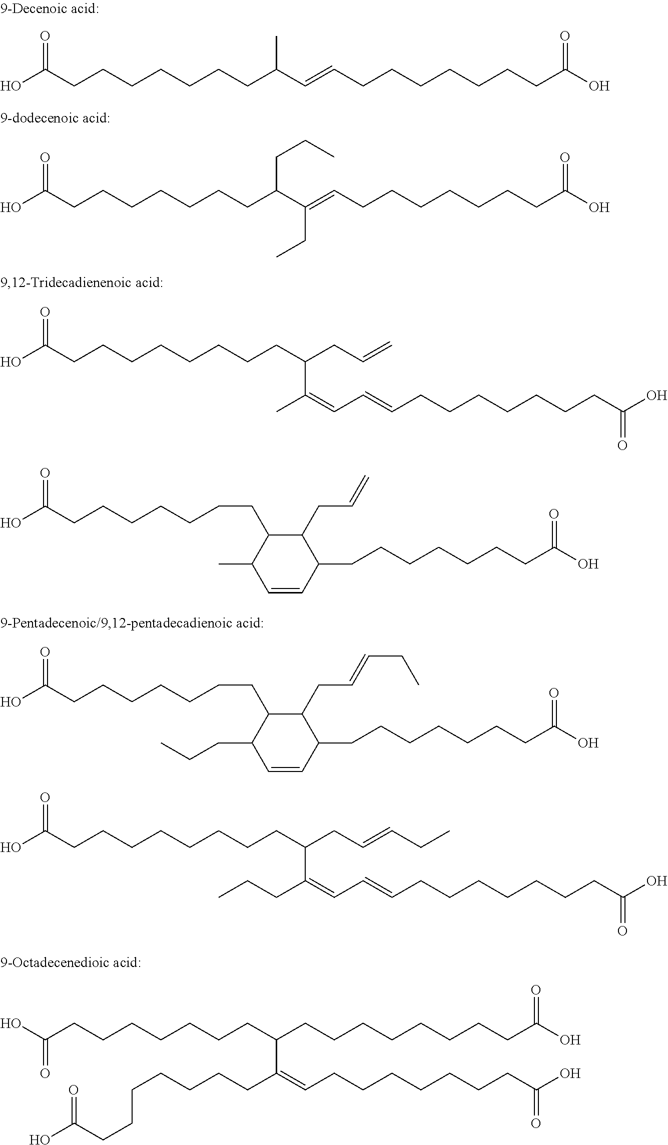

### Methods for Oligomerizing the Alkyl Ester or Carboxylic Acid

These oligomerization reactions can be carried out at 50° C. to 350° C., preferably 100° C. to 300° C., preferably 150° C. to 250° C., and more preferably about 160° C. to 220° C. The reaction pressure can be atmospheric pressure to 500 psi. Atmospheric pressure or slightly above, up to 150 psi are convenient operating pressures. The reaction may optionally be carried out in the presence of small amount of hydrogen gas to prevent or improve catalyst aging and promote long catalyst lifetime. The hydrogen pressure can range from 1 psi to 300 psi, alternatively, 5 psi to 250 psi, alternatively 30 psi to 200 psi, and alternatively 50 to 250 psi. Optimum amount of hydrogen is used to reduce coke or deposit formation on catalyst, to promote long catalyst life time without significant hydrogenation of mono-unsaturated fatty acids. Furthermore, the presence of hydrogen may slightly reduce the di- or poly-unsaturated fatty acid. Thus, the presence of hydrogen may reduce the cyclic dimer or oligomer formation. This is beneficial for production of high paraffinic hydrocarbons at the end of the conversion.

When solid catalyst is used, the reaction can be carried out in batch mode or in continuously stirred tank (CSTR) mode, or in fixed bed continuous mode. In one embodiment of a CSTR mode, a 600 mL Parr high pressure stainless-steel vessel can be used, which may be equipped with a mechanical stirrer, or an agitator to maintain the solids in suspension. In a batch or CSTR mode, the amount of catalyst used may vary from less than 0.01% to 30 wt % of the feed, preferably 1 to 20 wt %, depending on reaction time or conversion level. The reaction time or residence time may vary from 30 minutes to 50 hours, preferably 60 minutes to 10 hours, and most preferably about 2 hours to about 8 hours. In some instances, the vessel may be purged and sealed under nitrogen to withstand the steam pressure generated at the reaction temperatures.

Additionally, a catalyst modifier, i.e., an alkali or alkaline earth metal salt, may be added during the reaction. The modifier affects the selectivity of dimer in the reaction product. Additionally, when the modifier is lithium carbonate, lithium hydroxide or other lithium salts, the coloration of the product polymeric fatty acids is improved.

The crude product mixture of oligomers can be isolated by filtration to remove the product. The crude product mixture of oligomers generally refers to a product yield prior to further purification via conventional means (i.e. distillation). In some aspects, the crude product mixture can comprise from between about from about 18% to about 81% monomers of metathesized C₁₀-C₁₇ alkyl esters, from about 14% to about 46% dimers of metathesized C₁₀-C₁₇ alkyl esters, and from about 0% to about 18% trimers and/or higher unit oligomers of metathesized C₁₀-C₁₇ alkyl esters. In other aspects, the crude product mixture can comprise from about 30% to about 60% monomers of metathesized C₁₀-C₁₇ carboxylic acids, from about 30% to about 45% dimers of metathesized C₁₀-C₁₇ carboxylic acids, and from about 10% to about 25% trimers and/or higher unit oligomers of metathesized C₁₀-C₁₇ carboxylic acids.

The crude product mixture is then distilled to yield a purified product. The final conversion level varies from 10% to 100%, and alternatively from 20% to 90%. In some instances, high conversion minimizes problems associated with product separation. In some instances, partial conversion, such as 50 to 80%, is preferred to prevent excessive formation of undesirable byproducts. In some instances, the purified product comprises at least 93% dimers or trimers of metathesized C₁₀-C₁₇ alkyl esters, and in some instances, the purified product comprises at least 95% dimers or trimers of metathesized C₁₀-C₁₇ carboxylic acids. Optionally, the final dimerized or trimerized product or higher unit oligomerized product may be hydrogenated using known techniques, and such hydrogenated dimerized or trimerized or higher unit oligomerized product results in a lighter color than the non-hydrogenated dimerized or trimerized or higher unit oligomerized product. Additionally, the hydrogenated dimerized or trimerized or higher unit oligomerized product often exhibits improved oxidative stability. In some embodiments, the weight ratio of dimer to trimer ranges from 20:80 to 80:20, and preferably in an 80:20 ratio.

Such catalysts for oligomerization reactions are carried out with suitable catalysts at the aforementioned temperatures. Suitable catalysts include molecular sieves (both aluminosilicate zeolites and silicoaluminophosphates), amorphous aluminosilicates, cationic acidic clays, and other solid acid catalysts. Oligomerization may be achieved under cationic conditions and, in such embodiments, the acid catalyst may comprise a Lewis Acid, a Brønsted acid, or a combination thereof. The Lewis acids may include boron triflouride (BF₃), AlCl₃, zeolite, and the like, and complexes thereof, and combinations thereof. The Brønsted acids may include HF, HCl, phosphoric acid, acid clay, and the like, and combinations thereof. Oligomerization may be achieved using a promoter (e.g., an alcohol) or a dual promoter (e.g., an alcohol and an ester) as described U.S. Pat. Nos. 7,592,497 B2 and 7,544,850 B2, the teachings of which are incorporated by reference.

The oligomerization catalysts described herein may be supported on a support. For example, the catalysts may be deposited on, contacted with, vaporized with, bonded to, incorporated within, adsorbed or absorbed in, or on, one or more supports or carriers. The catalysts described herein may be used individually or as mixtures. The oligomerizations using multiple catalysts may be conducted by addition of the catalysts simultaneously or in a sequence.

According to International Zeolite Association (IZA) definitions, molecular sieves can be categorized according to the size of the pore opening. Examples of the molecular sieves can be of the large (>12-ring pore opening), medium (10-ring opening) or small (<8-ring pore opening) pore type. The molecular sieves structure types can be defined using three letter codes. Non-limiting examples of small pore molecular sieves include AEI, AFT, ANA, APC, ATN, ATT, ATV, AWW, BIK, CAS, CHA, CHI, DAC, DDR, EDI, ERI, GIS, GOO, KFI, LEV, LOV, LTA, MER, MON, PAU, PHI, RHO, ROG, SOD, THO, and substituted forms thereof. Non-limiting examples of medium pore molecular sieves include AFO, AEL, EUO, HEU, FER, MEL, MFI, MTW, MTT, MWW, TON, and substituted forms thereof. Non-limiting examples of large pore molecular sieves include BEA, CFI, CLO, DNO, EMT, FAU, LTL, MOR and substituted forms thereof. Other zeolite catalysts have a Si/Al molar ratio of greater than 2 and at least one dimension of the pore openings greater than or equal to 10-ring. Other solid zeolites include ZSM-5 (MFI), zeolite beta (BEA), USY family zeolites (FAU), MCM-22, MCM-49, MCM-56 (MWW). Mesoporous materials with pore openings greater than 20 angstroms, such as the MCM-41 family and SBA-15 type with aluminum incorporated into the structure and thus possess acidity, can also be used as oligomerization catalysts. Other zeolites may include 720KOA, 640HOA, and 690HOA available from Tosoh Corporation, or CP811C-300, CBV760, CBV901 available from Zeolyst International.

Other examples of clay catalysts include acidic, natural or synthetic Montmorillonites (including K10, KSF, K30), bentonite, silica clay, alumina clay or magnesia clay or silica-alumina clay. Other clay catalysts may include neutral clays (F-100, Ca—Mg bentonite), Fulcat 200, Fulcat 400, and acid treated clays, such as DC-2 (AmCol, acid treated Na—Mg bentonite). Other catalysts for the oligomerization processes may include toluene sulfonic acid catalyst, ion-exchange resin catalyst, and aluminum trichloride catalyst. Commercially available acidic forms of Filtrol clays are also suitable for this oligomerization process. Other solid acid catalysts, such as activated WOx/ZrO₂ catalysts, other metal oxides, Nafions or other acidic ion-exchanged resins, such as Dowex or Amberlyst cation exchanged are also suitable for the oligomerization reaction.

Optionally, the oligomerization reaction can also be catalyzed by homogeneous catalysts. Examples are hydrochloric acid, sulfuric acid, nitric acid, other small carboxylic acids or BF₃, promoted BF₃ catalysts, AlCl₃ or promoted AlCl₃ catalysts. When these homogeneous catalysts are used, 0.1 wt % to 10 wt % of catalyst may be used. Reaction temperatures for homogeneous acid catalyzed reaction range from 20° C. to 150° C. At the end of the reaction, these homogeneous acid catalysts are removed by aqueous wash or by adsorption by solid sorbents. The oligomerization reaction can also be catalyzed by the carboxylic acid itself when no other catalysts are added.

As known by a person skilled in the art, alkyl esters and carboxylic acids may be oligomerized (including dimerization) via known techniques. A variety of dimerization processes have been described. For example, in Kirk-Othmer: Encyclopedia of Chemical Technology, 3^{rd} Ed., vol. 7, Dimer acids, p. 768, a method is presented for producing dimeric acids from unsaturated carboxylic acids with a radical reaction using a cationic catalyst, the reaction temperature being 230° C. In addition to acyclic unsaturated dimeric acid as the main product, mono- and bi-cyclic dimers are also formed. In Koster R. M. et al., Journal of Molecular Catalysis A: Chemical 134 (1998) 159-169, oligomerization of carboxylic acids, carboxylic acid methyl esters, and synthetic alcohols and olefins is described, yielding corresponding dimers. Additional processes to oligomerize alkyl esters and carboxylic acids have also been described in U.S. Pat. Nos. 2,793,219, 2,793,220, 2,955,121, 3,632,822, 3,422,124 and 4,776,983, 4,895,982, and 5,001,260, the contents of each of which are incorporated by reference herein in their entireties.

### Uses/Applications for Oligomerized Alkyl Esters and Carboxylic Acids

The oligomerized alkyl esters and/or oligomerized carboxylic acids, or derivatives therefrom, may be used in various industrial or commercial applications. As used in this context, “derivatives” includes not only chemical compositions or materials resulting from the reaction of oligomerized alkyl esters and/or oligomerized carboxylic acids with at least one other reactant to form a reaction product, and further downstream reaction products of those reaction products as well.

The end uses for oligomerized alkyl esters and/or oligomerized carboxylic acids, or derivatives therefrom, include solid and liquid polyamide resins, epoxy and polyester resins for use, in thermographic inks and coatings for plastic films, papers, and paperboard. The oligomerized alkyl esters and/or oligomerized carboxylic acids, or derivatives therefrom, may be incorporated into various formulations and used as lubricants, functional fluids, fuels and fuel additives, additives for such lubricants, functional fluids and fuels, plasticizers, asphalt additives, friction reducing agents, antistatic agents in the textile and plastics industries, flotation agents, gelling agents, epoxy curing agents, corrosion inhibitors, pigment wetting agents, in cleaning compositions, plastics, coatings, adhesives, surfactants, emulsifiers, skin feel agents, film formers, rheological modifiers, solvents, release agents, conditioners, and dispersants, hydrotropes, etc. Where applicable, such formulations may be used in end-use applications including, but not limited to, personal care, as well as household and industrial and institutional cleaning products, oil field applications, gypsum foamers, coatings, adhesives and sealants, agricultural formulations, to name but a few. Thus, the oligomerized alkyl esters and/or oligomerized carboxylic acids, or derivatives therefrom may be employed as or used in applications including, but not limited to bar soaps, bubble baths, shampoos, conditioners, body washes, facial cleansers, hand soaps/washes, shower gels, wipes, baby cleansing products, creams/lotions, hair treatment products, anti-perspirants/deodorants, enhanced oil recovery compositions, solvent products, gypsum products, gels, semi-solids, detergents, heavy duty liquid detergents (HDL), light duty liquid detergents (LDL), liquid detergent softener antistat formulations, dryer softeners, hard surface cleaners (HSC) for household, autodishes, rinse aids, laundry additives, carpet cleaners, softergents, single rinse fabric softeners, I&I laundry, oven cleaners, car washes, transportation cleaners, drain cleaners, defoamers, anti-foamers, foam boosters, anti-dust/dust repellants, industrial cleaners, institutional cleaners, janitorial cleaners, glass cleaners, graffiti removers, concrete cleaners, metal/machine parts cleaners, pesticide emulsifiers, agricultural formulations and food service cleaners.

The oligomerized alkyl esters and/or oligomerized carboxylic acids, or derivatives therefrom may be incorporated into, for example, various compositions and used as lubricants, functional fluids, fuels, additives for such lubricants, functional fluids and fuels, plasticizers, asphalt additives and emulsifiers, friction reducing agents, plastics, coatings, adhesives, surfactants, emulsifiers, skin feel agents, film formers, rheological modifiers, biocides, biocide potentiators, solvents, release agents, conditioners, and dispersants, etc. Where applicable, such compositions may be used in end-use applications including, but not limited to, personal care liquid cleansing products, conditioning bars, oral care products, household cleaning products, including liquid and powdered laundry detergents, liquid and sheet fabric softeners, hard and soft surface cleaners, sanitizers and disinfectants, and industrial cleaning products, emulsion polymerization, including processes for the manufacture of latex and for use as surfactants as wetting agents, dispersants, solvents, and in agriculture applications as formulation inerts in pesticide applications or as adjuvants used in conjunction with the delivery of pesticides including agricultural crop protection turf and ornamental, home and garden, and professional applications, and institutional cleaning products. They may also be used in oil field applications, including oil and gas transport, production, stimulation and drilling chemicals and reservoir conformance and enhancement, organoclays for drilling muds, specialty foamers for foam control or dispersancy in the manufacturing process of gypsum, cement wall board, concrete additives and firefighting foams, paints and coatings and coalescing agents, paint thickeners, adhesives, or other applications requiring cold tolerance performance or winterization (e.g., applications requiring cold weather performance without the inclusion of additional volatile components).

The oligomerized alkyl esters and/or oligomerized carboxylic acids, or derivatives therefrom may be used in all types of adhesives, sealants and coatings, tackifiers, solvents, tire and rubber modification for tread and tire enhancement, air care (soy gels, air freshener gels) cutting, drilling and lubricant oils, linoleum binders, paper sizing, clear candles, ink resins and binders, road marking resins, reflective road marking through incorporation of glass beads on road markings, pigment coatings and as an end block reinforcing resin in styrene-isoprene-styrene (SIS) and styrene-butadiene-styrene (SBS) block copolymers for pressure sensitive adhesives.

The formulations mentioned above commonly contain one or more additional components for various purposes, such as surfactants, anionic surfactants, cationic surfactants, ampholtyic surfactants, zwitterionic surfactants, mixtures of surfactants, builders and alkaline agents, enzymes, adjuvants, fatty acids, odor control agents and polymeric suds enhancers, and the like.

The following examples merely illustrate the invention. The skilled person will recognize many variations that are within the spirit of the invention and scope of any current or future claims.

### EXAMPLES

### Acid-Catalyzed Oligomerization of 9-DAME(methyl-9-decenoate)

### Reaction Conditions and Catalysts

Screening reactions were carried out to evaluate the acid catalysts under various operating conditions. The experiments were performed in closed reaction vessels under nitrogen at 3-10 g scale at 160-260° C. using 5-30 wt % catalyst for four to nine hours. For higher temperature reactions, methanol was added to reduce lactone formation. Exemplary screening reactions are provided below with the resultant products reported in area percent. Results of the preliminary catalyst screenings are found in Table 1. Table 2 contains the results of larger scale reactions that were carried out in a 600 mL Parr reactor; exemplary procedures also are provided. The results of the optimization studies using K10 catalyst are provided in Table 3.

Clay type catalysts montmorillonite K10, KSF, K30, bentonite, and FLO supreme 8-81 (bleaching clay) were obtained from Sigma Aldrich. The zeolites 720KOA, 640HOA and 690HOA were purchased from Tosoh, Japan. The zeolites CP811C-300, CBV760, CBV901 were purchased from Zeolyst International, USA. The soluble catalyst components, Amberlyst 15, and commercial 1-decene were purchased from Sigma Aldrich. The methyl 9-decenoate was made via the alkenolysis of an algal oil surrogate. Analyses were done by GC/MS using an Agilent model 7890A chromatograph.

Clay as Catalyst (No Solvent):

A mixture of 5 g of methyl-9-decenoate and 1 g MMT K10 (20% w/w) was heated at 190° C. in sealed vessel under a blanket of N₂ for 8 hours. Samples were taken at 4 hours, 6 hours and 8 hours. After 8 hours, the mixture was filtered through a syringe filter to give dark orange oil. GC/MS shows the following crude chemical composition (% area): monomer 42%, dimer 38%, trimer and higher oligomers 7%, 13% lactone.

Clay Catalyst with a Solvent:

A mixture of 5 g of methyl 9-decenoate, 0.5 g MMT KSF (10% w/w) and 0.1 mL (2% w/w) methanol was heated at 230° C. in sealed vessel under N₂ for 8 hours. Samples were taken at six and eight hours. After 8 hours, the mixture was filtered through a syringe filter to give dark orange oil. GC/MS shows the following crude chemical composition: 37% monomer, 43% dimer, 13% timer and higher oligomers, and 7% lactone.

Ion-Exchange Resin Catalyst:

A mixture of 10 g of methyl 9-decenoate and 1.25 g Amberlyst 15 was heated for four hours at 165° C. in a 100 ml single-neck round bottom flask equipped with a condenser and magnetic stir bar. Two grams of crude product was separated by silica gel column chromatography to give three fractions that were characterized by GC-MS: the first fraction contained 80% isomerized starting material, the second fraction was found to be 52% dimer, and the third fraction was found to be 54% lactone.

The crude filtered products from several methyl 9-decenoate oligomerizations reactions totaling 48 grams were combined and fractionated by vacuum distillation, yielding a first fraction (14.87 g) containing 90% isomerized monomer, a 7.9 g second fraction contains 59% lactone, and a 12.5 g third fraction containing 60% dimer and 40% higher oligomers. The lactone structure was confirmed by ¹H NMR.

Toluene Sulfonic Acid (p-TSA) Catalyst:

A mixture of 3 g of methyl 9-decenoate and 0.15 g p-TSA was heated at 100° C. for four hours under N₂ in sealed vessel. Thin layer chromatography showed mostly unreacted starting material. The mixture was heated for an additional five hours at 160° C., after which the GC/MS indicated the following composition: 73% unreacted 9-DAME, 25% isomers of 9-DAME, and 2% other by-products.

Aluminum Trichloride Catalyst:

A mixture of 10 g of methyl 9-decenoate and 0.4 g AlCl₃ was stirred at room temperature in a sealed vessel under nitrogen for 24 h. An aliquot that was analyzed by GC/MS showed only starting material. The reaction mixture was stirred at 60° C. for an additional 24 hours but no oligomers were found.

TABLE 1
Acid-Catalyzed Oligomerization of 9-DAME -- Preliminary
Catalyst Screening.

| | | | | | | | | Lactone |
|---|---|---|---|---|---|---|---|---|
| | | | | | | | | and Other |
| | Catalyst | | | | Dimers | | Higher | By- |
| | Loading | Temp | Time | Monomers | (% | Dimer/Monomer | Oligomers | Products |
| Catalyst | (% wt/wt) | (° C.) | (hr) | (% area) | area) | Ratio | (% area) | (% area) |
| MMT K10 | 8.5 | 190 | 8 | 59.0 | 25.0 | 0.42 | 6.0 | 10.0 |
| MMT K10 | 10 | 190 | 4 | 69.0 | 21.0 | 0.30 | 0.0 | 10.0 |
| MMT K10 | 20 | 190 | 4 | 57.0 | 27.0 | 0.47 | 8.0 | 8.0 |
| MMT K10 | 20 | 190 | 6 | 49.0 | 31.5 | 0.64 | 9.0 | 10.5 |
| MMT K10 | 20 | 190 | 8 | 42.0 | 38.0 | 0.90 | 7.0 | 13.0 |
| MMT K10 | 30 | 190 | 8 | 30.0 | 42.0 | 1.40 | 18.0 | 10.0 |
| MMT K10 | 20 | 200 | 8 | 33.0 | 45.5 | 1.38 | 7.5 | 18 (4.8) |
| MMT K10 | 20 | 200 | 2 | 46.0 | 36.6 | 0.80 | 7.0 | 10.4 (5) |
| MMT K10 | 20 | 200 | 4 | 36.0 | 42.5 | 1.18 | 8.6 | 12.9 (5) |
| MMT K30 | 20 | 190 | 8 | 65.0 | 18.0 | 0.28 | 0.0 | 17 (8) |
| MMT KSF | 10 | 240 | 6 | 84.0 | 11.0 | 0.13 | 0.0 | 5.0 |
| (no | | | | | | | | |
| MeOH) | | | | | | | | |
| MMT KSF | 10 | 240 | 6 | 62.0 | 20.0 | 0.32 | 4.0 | 14.0 |
| (2% | | | | | | | | |
| MeOH) | | | | | | | | |
| MMT KSF | 10 | 190 | 8 | 66.0 | 19.0 | 0.29 | 6.0 | 9.0 |
| (no | | | | | | | | |
| MeOH) | | | | | | | | |
| MMT KSF | 20 | 190 | 2 | 63.0 | 21.4 | 0.34 | 0.0 | 15.6 |
| (no | | | | | | | | |
| MeOH) | | | | | | | | |
| MMT KSF | 20 | 190 | 4 | 53.0 | 27.0 | 0.51 | 0.0 | 20.0 |
| (no | | | | | | | | |
| MeOH) | | | | | | | | |
| MMT KSF | 20 | 190 | 8 | 37.0 | 30.0 | 0.81 | 10.0 | 23.0 |
| (no | | | | | | | | |
| MeOH) | | | | | | | | |
| MMT KSF | 10 | 230 | 6 | 44.7 | 37.6 | 0.84 | 10.2 | 7.5 |
| (2% | | | | | | | | |
| MeOH) | | | | | | | | |
| MMT KSF | 10 | 230 | 8 | 37.0 | 43.0 | 1.16 | 13.0 | 7.0 |
| (2% | | | | | | | | |
| MeOH) | | | | | | | | |
| FLO | 10 | 190 | 8 | 75.0 | 18.5 | 0.25 | 6.0 | 0.5 |
| supreme | | | | | | | | |
| 8-81 clay | | | | | | | | |
| Bentonite | 10 | 190 | 8 | 93.0 | 5.0 | 0.05 | 2.0 | 0.0 |
| 720 KOA | 10 | 190 | 8 | 99.4 | 0.6 | 0.01 | 0.0 | 0.0 |
| 640 HOA | 10 | 190 | 8 | 89.6 | 10.0 | 0.11 | 0.0 | 0.4 |
| 690 HOA | 10 | 190 | 8 | 77.3 | 22.7 | 0.29 | 0.0 | 0.0 |
| 690 HOA | 10 | 230 | 6 | 73.6 | 23.0 | 0.31 | 0.0 | 3.4 |
| (no | | | | | | | | |
| (MeOH) | | | | | | | | |
| 690 HOA | 10 | 230 | 8 | 70.6 | 25.0 | 0.35 | 0.0 | 4.4 |
| (no | | | | | | | | |
| MeOH) | | | | | | | | |
| 690 HOA | 10 | 230 | 6 | 70.0 | 25.0 | 0.36 | 0.0 | 5.0 |
| (2% | | | | | | | | |
| MeOH) | | | | | | | | |
| 690 HOA | 10 | 230 | 8 | 68.5 | 25.5 | 0.37 | 0.0 | 6.0 |
| (2% | | | | | | | | |
| MeOH) | | | | | | | | |
| Amberlyst | 12.5 | 165-170 | 1 | 52.0 | 24.0 | 0.46 | 0.0 | 24.0 |
| 15 | | | | | | | | |
| Amberlyst | 12.5 | 170 | 3 | 18.7 | 34.0 | 1.82 | 18.0 | 29.3 |
| 15 | | | | | | | | |
| Amberlyst | 12.5 | 170 | 3 | 18.7 | 34.0 | 1.82 | 18.0 | 29.3 |
| 15 | | | | | | | | |
| Amberlyst | 12.5 | 170 | 3 | 37.0 | 33.0 | 0.89 | 0.0 | 30.0 |
| 15 | | | | | | | | |
| dried | | | | | | | | |
| Amberlyst | 5 | 130 | 2 | 81.0 | 14.0 | 0.17 | 0.0 | 5.0 |
| 15 | | | | | | | | |
| Amberlyst | 5 | 130 | 4 | 75.0 | 18.0 | 0.24 | 0.0 | 7.0 |
| 15 | | | | | | | | |
| Amberlyst | 10 | 130 | 2 | 66.0 | 24.0 | 0.36 | 0.0 | 10.0 |
| 15 | | | | | | | | |
| Amberlyst | 10 | 130 | 4 | 54.0 | 31.0 | 0.57 | 2.5 | 12.5 |
| 15 | | | | | | | | |
| Amberlyst | 10 | 165 | 1 | 56.0 | 24.0 | 0.43 | 2.0 | 18.0 |
| 15 | | | | | | | | |
| Amberlyst | 10 | 165 | 2 | 50.0 | 24.0 | 0.48 | 3.0 | 23.0 |
| 15 | | | | | | | | |
| Amberlyst | 10 | 165 | 3 | 41.0 | 28.0 | 0.68 | 3.2 | 27.8 |
| 15 | | | | | | | | |
| Amberlyst | 10 | 165 | 4 | 34.0 | 31.0 | 0.91 | 5.7 | 29.3 |
| 15 | | | | | | | | |
| Amberlyst | 10 | 165 | 6 | 23.0 | 42.5 | 1.85 | 8.0 | 26.5 |
| 15 | | | | | | | | |
| CBV760 | 10 | 250 | 1 | 52.0 | 34.0 | 0.65 | | 14.0 |
| CBV760 | 10 | 250 | 2 | 31.0 | 43.5 | 1.40 | 0.0 | 25.5 (5.5) |
| CBV760 | 10 | 190 | 2 | 53.0 | 35.0 | 0.66 | | 12.0 |
| CBV760 | 10 | 190 | 4 | 48.0 | 41.5 | 0.86 | | 10.5 |
| CBV760 | 10 | 190 | 8 | 34.0 | 45.0 | 1.32 | 4.0 | 17 (7.5) |
| CBV901 | 10 | 250 | 1 | 80.0 | 16.7 | 0.21 | | 3.3 |
| CBV901 | 10 | 250 | 2 | 65.0 | 22.0 | 0.34 | | 13 (3) |
| CBV901 | 10 | 190 | 2 | 85.0 | 12.0 | 0.14 | | 3.0 |
| CBV901 | 10 | 190 | 4 | 81.0 | 10.0 | 0.12 | | 9.0 |
| CBV901 | 10 | 190 | 8 | 76.0 | 15.0 | 0.20 | | 9 (4) |
| CP811C- | 10 | 250 | 1 | 100.0 | 0.0 | 0.00 | | 0.0 |
| 300 | | | | | | | | |
| CP811C- | 10 | 250 | 2 | 97.8 | 2.2 | 0.02 | | 0.0 |
| 300 | | | | | | | | |
| CP811C- | 10 | 190 | 2 | 100.0 | 0.0 | 0.00 | | 0.0 |
| 300 | | | | | | | | |
| CP811C- | 10 | 190 | 4 | 100.0 | 0.0 | 0.00 | | 0.0 |
| 300 | | | | | | | | |
| CP811C- | 10 | 190 | 8 | 100.0 | 0.0 | 0.00 | | 0.0 |
| 300 | | | | | | | | |
| p-TSA | 2.5 | 100-165 | 6 | 100.0 | 0.0 | 0.0 | | 0.0 |
| p-TSA | 5 | 100-165 | 6 | 98.0 | 0.0 | 0.0 | | 2.0 |
| AlCl₃ | 4 | RT, | 24 + | 100.0 | 0.0 | 0.0 | 0 | 0.0 |
| | | then | 24 | | | | | |
| | | 60° C. | | | | | | |
| AlCl₃ | 79 | RT | 6 | 18.0 | 0.0 | 0.0 | 36.5 | 45.5 (no |
| | | | | | | | | lactone) |

### Acid-Catalyzed Oligomerization of 9-DAME(methyl-9-decenoate)—Larger Scale Reactions

Large Scale Reaction Using K10 at 200° C.:

Methyl 9-decenoate (9-DAME, 250 g) and 50 g (20% w/w) K10 clay were added to a glass liner and the liner was place into a 600 mL Parr reactor that was sealed and purged with N₂ for 15 minutes. An initial pressure of 8 psig N₂ was applied and the mixture heated to 200° C. while stirring at 600 rpm. The reaction mixture was stirred at 200° C. for 8 hours during which a pressure of 135 psig was achieved. The reaction mixture was diluted with ethyl acetate (1:1) and filtered under vacuum using a Buchner funnel fitted with filter paper. The residue was washed with 200 mL ethyl acetate and the ethyl acetate was stripped from the combined filtrate using a rotary evaporator, yielding 230 g of crude material. Vacuum distillation of the crude product at 190° C./20 torr yielded 93.3 g monomer and isomerized monomer. A second fraction that was distilled at 220° C./20 torr was found to be 8.8 g monomer and lactone. The distillation bottoms (127.5 g) have an iodine value of 90 and were found to be 94.8% dimers and trimers, 2.5% lactone, and 2.7% other by-products.

Large Scale Reaction Using KSF and Methanol:

Methyl 9-decenoate (9-DAME, 200 g), 20 g (10% w/w) KSF clay, 6 g methanol, and 0.2 g lithium carbonate were added a 600 mL Parr reactor that was sealed and purged with N₂ for 15 minutes. An initial pressure of 20 psig N₂ was applied and the mixture heated to 250° C. while stirring at 600 rpm. The reaction mixture was stirred at 250° C. for 6 hours during which it achieved a pressure of 370 psig. The reaction mixture was filtered under vacuum, the residue was washed with ethyl acetate and the ethyl acetate was stripped from the combined filtrate under vacuum, yielding 180 g of crude material. Vacuum distillation of the crude product at 190° C./20 torr yielded 60.8 g monomer and isomerized monomer. The distillation bottoms (113.7 g) have an iodine value of 90 and were found to be 94.7% dimers and trimers, 0.5% lactone, and 4.8% other by-products.

Large Scale Reaction Using CBV760:

Methyl 9-decenoate (9-DAME, 250 g) and 37.5 g (15% w/w) zeolite CBV760 were charged to a 600 mL Parr reactor that was sealed and purged with N₂ for 15 minutes. An initial pressure of 20 psig N₂ was applied and the mixture heated to 220° C. while stirring at 600 rpm. The reaction mixture was stirred at 220° C. for 6 hours. The reaction mixture was filtered under vacuum, the residue was washed with ethyl acetate, and the ethyl acetate was stripped from the combined filtrate under vacuum, yielding 220 g of crude material. Vacuum distillation of the crude product at 90° C./2 torr yielded 50 g monomer and isomerized monomer. A second fraction that was distilled at 165° C./2 torr was found to be 24 g monomer and lactone. The distillation bottoms (130 g) were found by GC/MS to be 99% dimers and turners.

Large Scale Reaction Using K10 at 220° C.:

Methyl 9-decenoate (9-DAME, 250 g) and 50 g (20% w/w) K10 clay were charged to a 600 mL Parr reactor that was sealed and purged with N₂ for 15 minutes. An initial pressure of 8 psig N₂ was applied and the mixture heated to 220° C. while stirring at 600 rpm. The reaction mixture was stirred at 220° C. for 8 hours during which samples were withdrawn every two hours. The reaction mixture was filtered under vacuum, the residue was washed with ethyl acetate, and the ethyl acetate was stripped under vacuum, yielding 220 g of crude material. Vacuum distillation of the crude product at 140° C./2 torr yielded 58 g monomer and isomerized monomer. A second fraction that was distilled at 200° C./2 torr was found to be 18.8 g monomer and lactone. The distillation bottoms (143 g) were found to be 93.5% dimers and trimers, 0.9% lactone, and 5.6% other by-products.

Large Scale Reaction Using K10 for Six Hours at 220° C.:

Methyl 9-decenoate (9-DAME, 250 g) and 37.5 g (20% w/w) K10 clay were added to a 600 mL Parr reactor that was sealed and purged with N₂ for 15 minutes. An initial pressure of 8 psig N₂ was applied and the mixture heated to 220° C. while stirring at 600 rpm. The reaction mixture was stirred at 220° C. for 6 hours. The reaction mixture was filtered under vacuum, the residue was washed with ethyl acetate, and the ethyl acetate was stripped under vacuum, yielding 212 g of crude material. Vacuum distillation of the crude product at 140° C./25 torr yielded 60 g monomer and isomerized monomer. A second fraction that was distilled at 200° C./6 torr was found to be 34 g monomer, lactone, and acid. The distillation bottoms (116 g) were found to be 96.5% dimers and trimers, 0.7% lactone, and 2.5% decenoic acid.

TABLE 2
Acid-Catalyzed Oligomerization of 9-DAME - Larger Scale Reactions.

| | | | | | | | | Dimer + |
|---|---|---|---|---|---|---|---|---|
| | | | | | | | | Timer |
| | | | | | | | | (% area in |
| | | | | | | | | bottoms) |
| | Catalyst | | | | Dimers | | | after |
| | Loading | Temp | Time | Monomer | (% | Dimer/Monomer | Lactone | vacuum |
| Catalyst | (% wt/wt) | (° C.) | (hr) | (% area) | area) | Ratio | (% area) | stripping |
| K10 | 20 | 200 | 8 | 33.0 | 45.0 | 1.36 | 4.6 | 94.8 |
| KSF | 10 | 250 | 6 | 25.0 | 49.0 | 1.96 | 1.0 | 98.0 |
| CBV760 | 15 | 220 | 6 | 20.0 | 57.0 | 2.85 | 5.0 | 99.0 |
| K10 | 20 | 220 | 8 | 18.0 | 48.0 | 2.67 | 5.0 | 93.5 |
| K10 | 20 | 220 | 6 | 19.0 | 49.0 | 2.58 | nd | 96.5 |
| K10 | 15 | 220 | 6 | 66.0 | 24.0 | 0.36 | 4.0 | 96.5 |
| K10 | 15 | 250 | 6 | 43.0 | 37.0 | 0.86 | 2.7 | 93.0 |

### Preparation of Dimethyl Diester

Dimethyl 1, 20-Eicos-10-enedioate:

The linear C₂₀ dicarboxylate dimethyl ester (10-EDAME2) was prepared by self-metathesis of methyl 10-undecenoate for use as an analytical reference sample. A mixture of methyl 10-undecenoate (10 g, 50.5 mmol, Sigma-Aldrich) and C827 catalyst (5 mg, Materia) was heated in a closed vial at 60° C. for 2 h after which thin layer chromatography (TLC, 10% ethyl acetate/hexane) showed mostly starting material. An additional 10 mg of catalyst was added and the mixture was heated at 60° C. for two hours; TLC of an aliquot indicated some product had formed. Another 100 mg catalyst was added and mixture was stirred at 60° C. for an additional three hours. One gram of the reaction mixture was purified by column chromatography, yielding a 100 mg sample that was found by GC-MS to be 88% of the desired C₂₀ diester (parent m/z=368) and 11% the C₁₉ analogue (parent m/z=354) which was confirmed by ¹H NMR spectroscopy.

### Effect of Catalyst loading, Temperature, and Reaction Time on Product Distribution and Yield

The effects of catalyst loading, temperature and reaction time on conversion and selectivity were studied using K10 montmorillonite, an inexpensive, acid-treated clay having high surface area (230 m²/g) and large pore size (45-150 Å.) Small scale oligomerization reactions were carried out for two to eight hours using 10 to 30 wt % catalyst loadings at 160, 190, and 220° C. (as shown in Table 3). From these studies, the oligomerization yield is found to increase at higher temperatures, higher catalyst loadings, and longer reaction times.

Guided by these results, a larger scale reaction (250 g 9-DAME) was performed in a Parr reactor with overhead stirring and the reaction was monitored every two hours (see Table 4). Fractional distillation of the resulting 220 g crude product at 140° C./2 torr yielded 58 g monomer and isomerized monomer. A second fraction that was distilled at 200° C./2 torr was found to be 18.8 g monomer and lactone. The distillation bottoms (143 g) were found to be 93.5% dimers and trimers, 0.9% lactone, and 5.6% other by-products. The GC/MS of the orange-brown bottoms (Figure) shows mostly dimer and trimer in about an 80/20 ratio.

TABLE 3
Oligomerization of 9-DAME using K10 Clay

| | Catalyst | | | | | Dimer/ |
|---|---|---|---|---|---|---|
| | Loading | Temp | Time | Monomers | Dimers | Monomer |
| Catalyst | (% wt/wt) | (° C.) | (hr) | (% area) | (% area) | Ratio |
| K10 | 10 | 160 | 2 | 100.00 | 0.00 | 0.00 |
| K10 | 10 | 160 | 4 | 94.50 | 5.50 | 0.06 |
| K10 | 10 | 160 | 6 | 93.00 | 6.50 | 0.07 |
| K10 | 10 | 160 | 8 | 91.00 | 8.00 | 0.09 |
| K10 | 15 | 160 | 2 | 100.00 | 0.00 | 0.00 |
| K10 | 15 | 160 | 4 | 93.50 | 6.50 | 0.07 |
| K10 | 15 | 160 | 6 | 90.00 | 9.00 | 0.10 |
| K10 | 15 | 160 | 8 | 89.00 | 9.50 | 0.11 |
| K10 | 20 | 160 | 2 | 95.00 | 5.00 | 0.05 |
| K10 | 20 | 160 | 4 | 88.00 | 10.30 | 0.12 |
| K10 | 20 | 160 | 6 | 88.00 | 10.00 | 0.11 |
| K10 | 20 | 160 | 8 | 85.00 | 12.00 | 0.14 |
| K10 | 30 | 160 | 2 | 89.60 | 9.50 | 0.11 |
| K10 | 30 | 160 | 4 | 86.00 | 12.00 | 0.14 |
| K10 | 30 | 160 | 6 | 83.00 | 13.00 | 0.16 |
| K10 | 30 | 160 | 8 | 81.00 | 14.00 | 0.17 |
| K10 | 10 | 190 | 2 | 89.00 | 9.00 | 0.10 |
| K10 | 10 | 190 | 4 | 86.00 | 12.00 | 0.14 |
| K10 | 10 | 190 | 6 | 78.00 | 13.00 | 0.17 |
| K10 | 10 | 190 | 8 | 57.50 | 14.00 | 0.24 |
| K10 | 15 | 190 | 2 | 87.00 | 11.00 | 0.13 |
| K10 | 15 | 190 | 4 | 83.00 | 13.00 | 0.16 |
| K10 | 15 | 190 | 6 | 83.00 | 13.75 | 0.17 |
| K10 | 15 | 190 | 8 | 75.00 | 18.00 | 0.24 |
| K10 | 20 | 190 | 2 | 82.00 | 14.00 | 0.17 |
| K10 | 20 | 190 | 4 | 78.50 | 14.50 | 0.18 |
| K10 | 20 | 190 | 6 | 69.00 | 18.70 | 0.27 |
| K10 | 20 | 190 | 8 | 66.00 | 22.00 | 0.33 |
| K10 | 30 | 190 | 2 | 77.00 | 15.00 | 0.19 |
| K10 | 30 | 190 | 4 | 71.00 | 16.50 | 0.23 |
| K10 | 30 | 190 | 6 | 65.00 | 19.50 | 0.30 |
| K10 | 30 | 190 | 8 | 60.00 | 23.50 | 0.39 |
| K10 | 10 | 220 | 2 | 67.50 | 15.40 | 0.23 |
| K10 | 10 | 220 | 4 | 66.00 | 19.00 | 0.29 |
| K10 | 10 | 220 | 6 | 57.00 | 20.50 | 0.36 |
| K10 | 10 | 220 | 8 | 52.00 | 23.00 | 0.44 |
| K10 | 15 | 220 | 2 | 64.00 | 18.50 | 0.29 |
| K10 | 15 | 220 | 4 | 59.00 | 19.50 | 0.33 |
| K10 | 15 | 220 | 6 | 45.00 | 26.50 | 0.59 |
| K10 | 15 | 220 | 8 | 42.00 | 28.50 | 0.68 |
| K10 | 20 | 220 | 2 | 57.00 | 20.50 | 0.36 |
| K10 | 20 | 220 | 4 | 54.00 | 21.50 | 0.40 |
| K10 | 20 | 220 | 6 | 45.00 | 29.50 | 0.66 |
| K10 | 20 | 220 | 8 | 40.00 | 32.00 | 0.80 |
| K10 | 30 | 220 | 2 | 50.00 | 26.00 | 0.52 |
| K10 | 30 | 220 | 4 | 44.00 | 27.00 | 0.61 |
| K10 | 30 | 220 | 6 | 42.00 | 31.50 | 0.75 |
| K10 | 30 | 220 | 8 | 35.00 | 37.00 | 1.06 |

TABLE 4
Large Scale Oligomerization of 9-DAME
at 220° C. using 20 wt % K10 Clay.

| | | | Dimer/ |
|---|---|---|---|
| Time | Monomers | Dimers | Monomer |
| (hr) | (% area) | (% area) | Ratio |
| 2 | 30 | 44 | 1.47 |
| 4 | 21 | 49 | 2.33 |
| 6 | 19 | 49 | 2.58 |
| 8 | 18 | 48 | 2.67 |

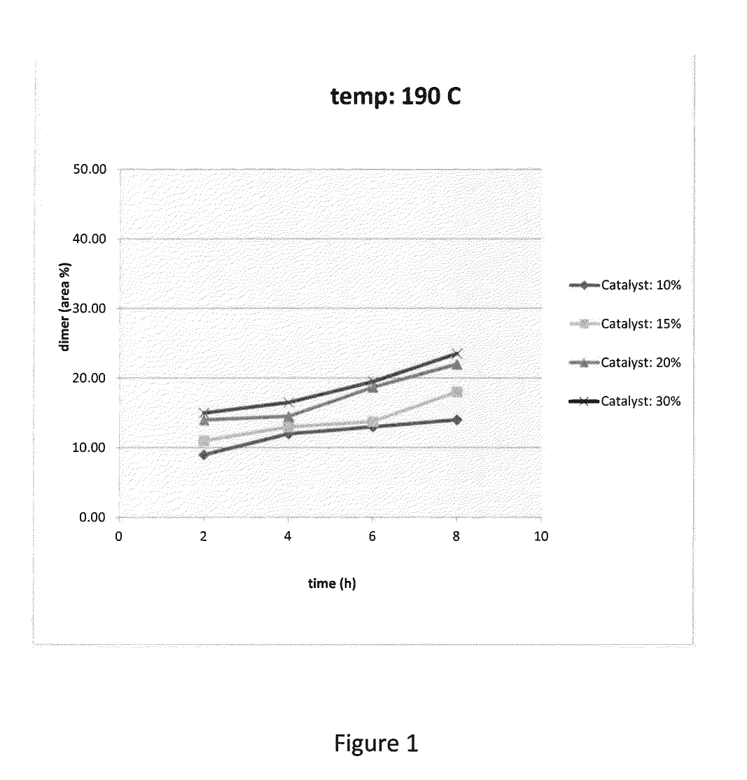

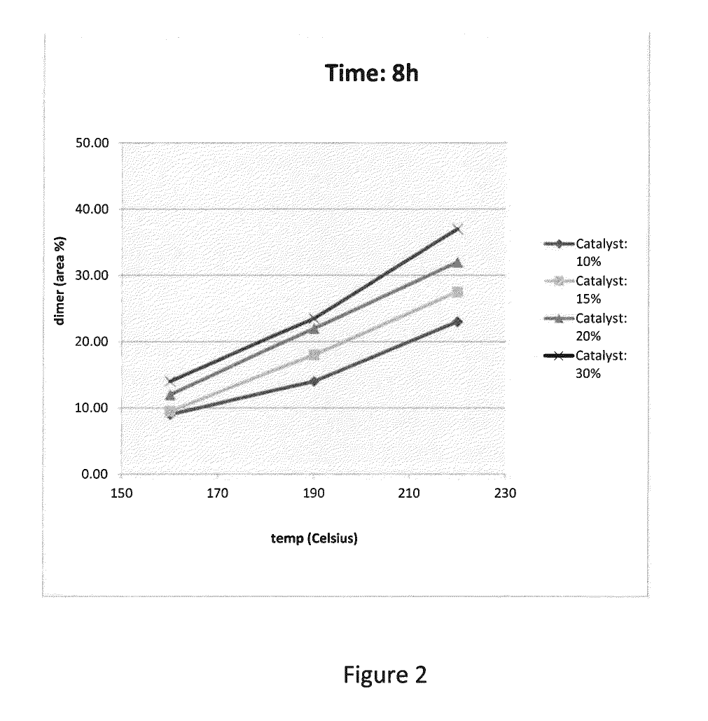

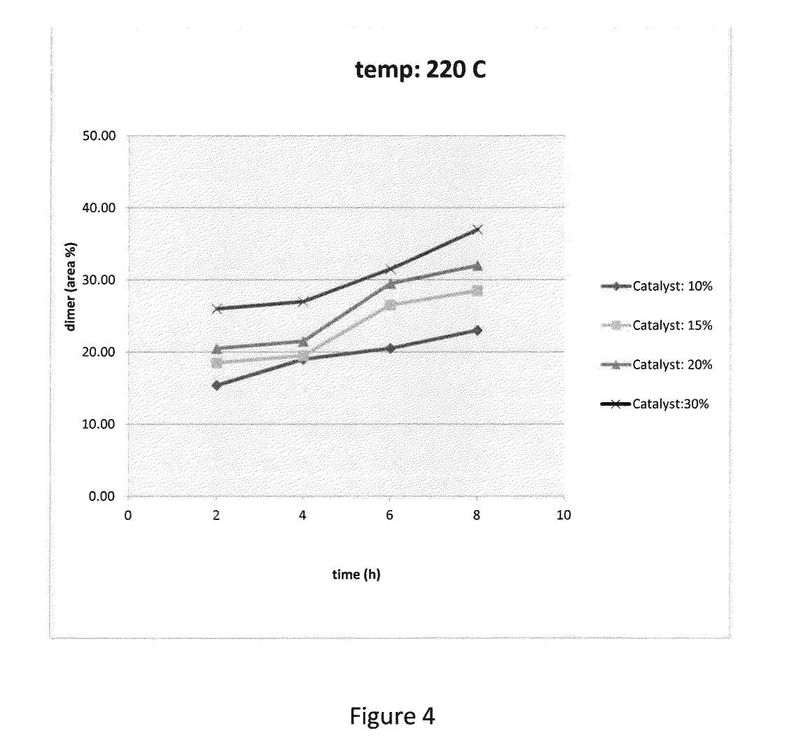

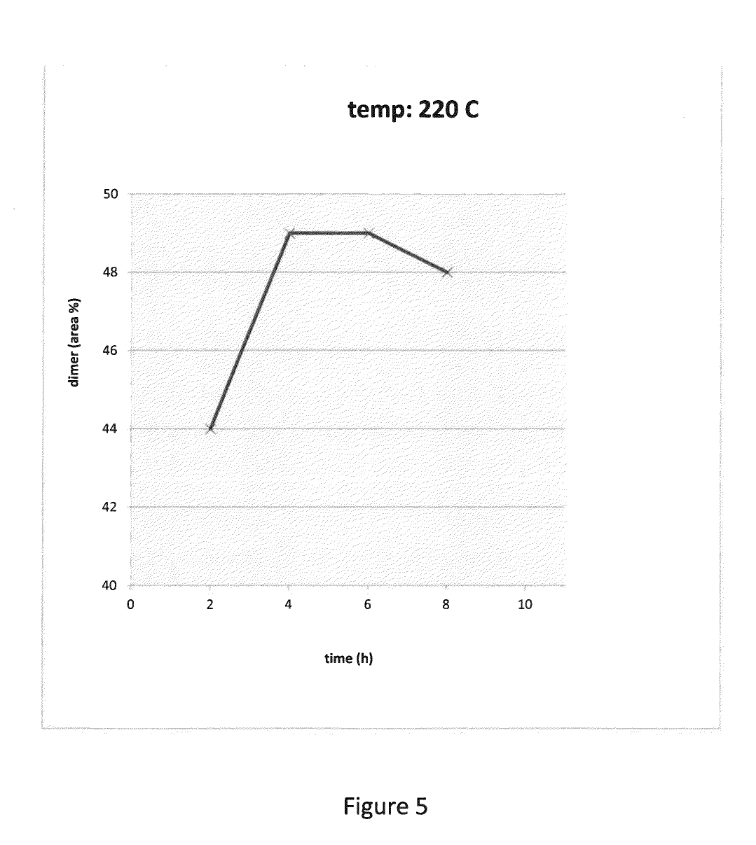

The rate of oligomerization was higher with higher temperature and high catalyst loading for the same reaction time. At low catalyst loading (10% w/w), the rate of reaction was very low (FIG. 1). However, at higher loading (20% w/w), although the conversion increased, the selectivity toward dimers decreased with more trimer forming (FIG. 5). Higher temperature increased the dimer, also the turner formation for the tested catalyst load and reaction times (FIG. 2). Longer reaction time only moderately increased conversion ratio for tested catalyst loads (FIG. 1 and FIG. 3). Longer reaction time substantially increased conversion ratio for tested catalyst loads at 220° C. (FIG. 4).

### Acid Catalyzed Oligomerization of 9DA (9-decenoic acid)

Experimental Procedure Using K10:

A mixture of 300 g of 9-decenoic acid, 36 g (12% w/w) Montmorillonite K10, water 3 g (0.1% w/w) and lithium carbonate (0.3 g, 0.1% w/w) was loaded in a 600 mL Parr reactor, sealed, purged with N2 for 30 minutes, an initial pressure of N2 (30 psi) was applied and the mixture was heated to 250° C. under 600 rpm stirring. The reaction mixture reached the desired temperature and then stirred at this temperature for 4 hours. After 4 hrs, the reaction mixture was cooled to 60° C. and transferred to a glass container. Vacuum filtration of the mixture to remove the catalyst was done using Buchner funnel and a pad of celite. To speed up the filtration, the reaction mixture was diluted with ethyl acetate (1:1). Catalyst was washed with ethyl acetate (200 ml) to maximize recovery. Ethyl acetate was removed using a rotary evaporator. Separation of monomer, lactone and dimer/higher oligomer was done by vacuum distillation.

Distillation of 280 g crude material (93% mass recovery) gave 179 g product, 59.6% yield. At 4 mmHg, 200° C., 16 g fraction 1 (rearranged monomeric starting material) was separated. At 2 mmHg, 250° C., 74 g fraction 2 (some rearranged starting material and lactone) was separated. The distillation residue (179 g) was a mixture of dimers and trimers, after the derivatization of sample: dimer/trimer 83.14% (combined % by GC/FID), 16.86% monomer. Acid number=294 mg KOH/g sample. Other characterization method: FTIR, SEC/GPC.

Use of Various Catalysts:

Several reaction conditions for 9-decenoic acid oligomerization were investigated in order to minimize the trimer/tetramer formation. The rationale in these experiments related to driving selectivity for either monomer (branched isomers) and/or dimer (building block for different applications). The catalysts investigated were neutral clay (F-100, BASF, Ca—Mg bentonite), Fulcat 200, Fulcat 400 and acid treated clays: DC-2 (AmCol, acid treated Na—Mg bentonite). The final composition was compared with the composition from K10 catalyzed oligomerization by GC/FID after derivatization. Reactions were run using 600 ml Parr reactor, same conditions: catalyst load (4.5% by wt), 250° C., 4 hours. Crude composition was analyzed by GC/FID. Results of the catalyst screening are shown in Table 5 below.

TABLE 5

| | Conversion %, | Ratio Dimer/trimer + |
|---|---|---|
| Catalyst | based on GC/FID | higher |
| K10, acid treated | 34 | 4.5/1 |
| DC-2, acid treated | 48 | 2.25/1 trimer and higher |
| F100, neutral | 55 | 2.4/1 trimer and higher |
| Fulcat 200, acid treated | 56 | 1.8/1 |
| Fulcat 400, acid treated | 59 | 1.8/1 |

From Table 5, K10 provided higher selectivity toward dimers (high ratio dimer/trimer), but the lowest conversion. Fulcat 200 and 400 provided the highest conversion, but the lowest selectivity. Based on screening results, F100 provided a balanced conversion and selectivity, and was investigated further as shown below.

Experimental Procedure Using F100:

A mixture of 1200 g of 9-decenoic acid, 54 g (4.5% w/w) neutral clay F100 (BASF), water 24 g (2% w/w) and lithium carbonate (1 g, 0.08% w/w) was loaded in a 2 L Parr reactor, sealed, purged with N2 for 30 minutes, an initial pressure of N2 (30 psi) was applied and the mixture was heated to 250° C. under 600 rpm stirring. The reaction mixture reached the desired temperature and then stirred at this temperature for 4 hours. After 4 hrs, the reaction mixture was cooled to 60° C. and transferred to a flask. The mixture was treated with 11 g of 75% phosphoric acid at 130-135° C. for one hour to convert the soaps or interesters to free acid and remove color. Optionally, a bleaching clay could be used to remove color.

The mixture was cooled to 80° C. and vacuum filtered using Buchner funnel (medium core) and a pad of celite. This was the main filtrate. Catalyst was washed with toluene several times to maximize recovery (and use of pressure filter might increase mass recovery). The second filtrate was concentrated using a rotary evaporator to remove toluene used for catalyst washing. Combined filtrates (1100 g, 91.66% mass recovery) was fractionated using vacuum distillation. Crude product composition by GC/FID (area %) after derivatization was: Monomer: 35.5, Dimer: 42.2, Trimer: 20, Tetramer: 2.3.

Distillation of 1100 g crude material gave 620 g product, 50% yield, based on mass recovered. At 2 mmHg, 150° C., 300 g fraction 1 (rearranged monomeric starting material) was separated. At 2 mmHg, 200° C., 140 g fraction 2 (some rearranged starting material and lactone) was separated. The distillation residue (550 g) is a mixture of monomers, dimers and trimers. Based on GC/FID (area %) after sample was derivatized, the product composition is: monomer 4.3%, dimer 64.5%, trimer and higher 31.2%. Yield can be calculated as: % residue=100%×residue (wt)/[residue (wt)+distillate (wt)].

### Isolation and Characterization of High Dibasic Content of C20 Dimer Acid

Experimental Procedure Using K10:

A mixture of 1200 g of 9-decenoic acid, 96 g (8% w/w) Montmorillonite K10, water 9.6 g (0.8% w/w) and lithium carbonate (1.2 g, 0.1% w/w) was loaded in a 2 L Parr reactor, sealed, purged with N2 for 30 minutes, an initial pressure of N2 (30 psi) was applied and the mixture was heated to 250° C. under 600 rpm stirring. The reaction mixture reached the desired temperature and then stirred at this temperature for 4 hours. After 4 hrs, the reaction mixture was cooled to 60° C. and transferred to a flask. The mixture was treated with 11 g of 75% phosphoric acid at 130-135° C. for one hour to convert the soaps or interesters to free acid and remove color. Optionally, a bleaching clay could be used to remove color.

The mixture was cooled to 80° C. and vacuum filtered using Buchner funnel (medium core) and a pad of celite. This is the main filtrate. Catalyst was washed with toluene several times to maximize recovery. The second filtrate was concentrated using a rotary evaporator. Combined filtrates (1100 g, 91.66% mass recovery) was fractionated using vacuum distillation. Crude composition after derivatization: monomer 55.75%, dimer 32.6%, turner 10.65%, tetramer 0.76%.

Distillation of 1100 g crude material gave 540 g product, 49% yield, based on mass recovered. At 2 mmHg, 190° C., 530 g fraction 1 (rearranged monomeric starting material) was separated. At 2 mmHg, 200° C., 540 g fraction 2 (some rearranged starting material and lactone) was separated.

The distillation residue (540 g) is a mixture of monomers, dimers and trimers. Based on GC/FID (area %) after sample was derivatized, the product composition is: monomer 5.2%, dimer 65%, trimer and higher 29.5%.

The synthesized C20 dimer acid composition comparison to commercial dimer acid compositions and literature are shown in Table 6 below:

TABLE 6

| | Synthesized | Kirk Othmer | Arizona Chemical |
|---|---|---|---|
| | C20 dimer | Encyclopedia | C36 dimer |
| Monomer | 1-5% | 1-5% | 2 max. |
| Dimer | 65-70% | 82-83% | 79-85% |
| Trimer | 20-25% | 14-16% | 15-19% |
| Tetramer | 2-5% | 1-5% | 0 |

Separation of the synthesized high purity C20 dimer was done by wipe film evaporation in two passes. Distilled dimer with a high dibasic content compared to commercial C36 dimer in Table 7 below. Composition was determined by GC/FID after derivatization.

TABLE 7

| | Synthesized | |
|---|---|---|
| | C20 dimer | Empol 1061 |
| Monomer | 0.6 | 4.6 |
| Dimer | 99.2 | 93.8 |
| Trimer | 0.2 | 1.6 |

Certain physical properties of synthesized high purity C20 dimer was shown in Table 8 below.

TABLE 8

| | | Synthesized | |
|---|---|---|---|
| Test | Method | C20 dimer | Empol 1061 |
| Acid number | ASTM D-664, mg/g | 293 | 196 |
| Iodine value | AOCS Cd 1d-92, | 117 | 25 |
| | cg/g | | |
| Color | Gardner | 5.3 | 4.6 |
| Viscosity, 25□ C. | Brookfield, cP | 1682 | 7030 |

### Oligomerization of Mixture of C13, C14, and C15, Fatty Acid Methyl Esters

### Experimental Procedure Using K10:

A mixture of 300 g of fatty methyl esters and 45 g (15% w/w) Montmorillonite K10 was loaded in a 600 mL Parr reactor, sealed, purged with N2 for 30 minutes, an initial pressure of N2 (30 psi) was applied and the mixture was heated to 250° C. under 600 rpm stirring. The reaction mixture reached the desired temperature and then stirred at this temperature for 4 hours. After 4 hrs, the reaction mixture was cooled to 60° C. and transferred to a glass container. Vacuum filtration of the mixture to remove the catalyst was done using Buchner funnel and a pad of basic celite. Catalyst was washed with ethyl acetate (200 ml) to maximize recovery. Ethyl acetate was removed using a rotary evaporator. Separation of monomer, lactone and dimer/higher oligomer was done by vacuum distillation.

Distillation of 295 g crude material (95% mass recovery) gave 170 g product, 56.6% yield. At 4 mmHg, 200° C., 68 g fraction 1 (rearranged monomeric starting material) was separated. At 2 mmHg, 200° C., 53 g fraction 2 (some rearranged starting material and lactone) was separated. The distillation residue (170 g) is a mixture of monomers, dimers and trimers. Other characterization method: GC/FID, FTIR, SEC/GPC.

### Oligomerization of Mixture of 9DA and Oleic/LINOLEIC acid

Experimental Procedure Using K10:

A mixture of 9-decenoic acid (650 g, 3.8 mol), oleic/linoleic (550 g, 1.9 mol), Montmorillonite K10 (144 g, 12% w/w), lithium carbonate (1.2 g, 0.1%), water (12 g, 1%) was loaded in a 2 L Parr reactor, sealed, purged with N2 for 30 minutes, an initial pressure of N2 (30 psi) was applied and the mixture was heated to 250° C. under 600 rpm stirring. The reaction mixture reached the desired temperature and then stirred at this temperature for 4 hours. After 4 hrs, the reaction mixture was cooled to 60° C. and transferred to a glass container. The mixture was treated with 0.9% w/w 75% phosphoric acid at 135° C. for one hour to convert the soaps to free acid and remove color. Vacuum filtration of the mixture to remove the catalyst was done using Buchner funnel and a pad of celite. Catalyst was washed with toluene to maximize recovery. Toluene was removed using a rotary evaporator. Combined filtrate (1150 g, 95.8 mass recovery) was fractionated using vacuum distillation.

Distillation of 1150 g crude material (95.8% mass recovery) gave 900 g product, 75% yield. At 2 mmHg, 200° C., 130 g fraction 1 (rearranged monomeric starting material) was separated. At 2 mmHg, 230° C., 75 g fraction 2 (some rearranged starting material and lactone) was separated. The distillation residue (900 g) is a mixture of monomers, dimers and trimers. Other characterization method: GC/FID, FTIR, SEC/GPC.

What is claimed is:

1. A composition comprising a crude mixture of oligomers of C₁₀-C₁₇ alkyl esters, wherein the crude mixture comprises:
(a) from 18% to 81% as measured by GC/MS percent area, of monomers of C₁₀-C₁₇ alkyl esters,
(b) from 14% to 45% as measured by GC/MS percent area, of dimers of C10-C₁₇ alkyl esters,
(c) from 0% to 18% as measured by GC/MS percent area, of trimers and/or higher unit oligomers of C10-C₁₇ alkyl esters, and
(d) from 5% to 30% as measured by GC/MS percent area, of byproducts or lactone.

2. The composition of claim 1, wherein the C₁₀-C₁₇ alkyl esters comprises methyl-9-decenoate.

3. The composition of claim 1, wherein the monomers comprise a mixture of positionally and skeletally isomerized monomers of C₁₀-C₁₇ alkyl esters.

4. The composition of claim 1, wherein the crude mixture of oligomers are catalyzed by a catalyst selected from the group consisting of molecular sieves and ion exchange resins.

5. The composition of claim 1, wherein the crude mixture of oligomers is distilled to a purified product comprising at least 99% as measured by GC/MS percent area of dimers and trimers of C₁₀-C₁₇ alkyl esters.

6. The composition of claim 5, wherein the purified product comprising at least 99% dimers or trimers as measured by GC/MS percent area of C₁₀-C₁₇ alkyl esters is optionally hydrogenated.

7. A composition comprising a crude mixture of oligomers of methyl-9-decenoate, wherein the crude mixture comprises:
(a) from 31% to 53% as measured by GC/MS percent area, of monomers of methyl-9-decenoate,
(b) from 34% to 45% as measured by GC/MS percent area, of dimers of methyl-9-decenoate,
(c) from 0% to 8% as measured by GC/MS percent area, of trimers and/or higher unit oligomers of methyl-9-decenoate, and
(d) from 10.5% to 26.5% as measured by GC/MS percent area, of byproducts or lactone.

8. The composition of claim 7, wherein the monomers comprise a mixture of positionally and skeletally isomerized monomers of methyl-9-decenoate.

9. The composition of claim 7, wherein the crude mixture of oligomers are catalyzed by molecular sieves.

10. The composition of claim 7, wherein the crude mixture of oligomers is distilled to a purified product comprising at least 99% as measured by GC/MS percent area of dimers and trimers of methyl-9-decenoate.

11. A composition comprising a crude mixture of oligomers of C₁₀-C₁₇ carboxylic acids, wherein the crude mixture comprises:
(a) from 35.5% to 55.75% as measured by GC/FID percent area, of monomers of C₁₀-C₁₇ carboxylic acids,
(b) from 32.6% to 42.2% as measured by GC/FID percent area, of dimers of C₁₀-C₁₇ carboxylic acids, and
(c) from 11.4% to about 31.2% as measured by GC/FID percent area, of trimers and/or higher unit oligomers of C₁₀-C₁₇ carboxylic acids.

12. The composition of claim 11, wherein the C₁₀-C₁₇ carboxylic acids comprises 9-decenoic acid.

13. The composition of claim 11, wherein the crude mixture of oligomers are catalyzed by a clay catalyst.

14. The composition of claim 11, wherein the crude mixture of oligomers may be distilled to a purified product comprising at least 94.5% as measured by GC/FID percent area, of dimers and trimers of C₁₀-C₁₇ carboxylic acids.
